(19) United States
(12) Patent Application Publication
(10) Pub. No.: US 2020/0113957 A1
(43) Pub. Date: Apr. 16, 2020

(54) COMPOSITION BASED ON A NATURAL COMPOUND DERIVED FROM ANNONACEAE

(71) Applicant: AMESTAK S.A.S., Bogota (CO)

(72) Inventors: Juanita Gonzalez Aguilar, Bogota (CO); Eduardo Gonzalez Gutierrez, Bogota (CO); Catalina Gonzalez Cardona, Bogota (CO)

(21) Appl. No.: 16/499,852

(22) PCT Filed: Mar. 31, 2018
(86) PCT No.: PCT/IB2018/052245

§ 371 Date: Sep. 30, 2019

(30) Foreign Application Priority Data
Mar. 31, 2017 (CO) NC2017/0003236

(51) Int. Cl. A61K 36/185; A61K 9/06; A61K 9/00

(52) U.S. Cl. CPC A61K 36/185; A61K 9/0014; A61K 9/06

(57) ABSTRACT

The present invention relates to a composition comprising a natural active agent from the natural extract of Annona muricata with a specific mixture of excipients and water. The present invention provides a composition with a specific formulation design of Annona muricata leaves that achieves a proven effect in the treatment of cancer lesions.

### FIELD OF THE INVENTION

The present invention relates to the Pharmaceutical Chemistry sector, specifically to the design of compositions for application in the field of medicine and cosmetics.

The present invention corresponds to the development of a new associated natural formulation that acts as an anti-cancer agent.

### PRIOR ART

Alterations in human skin caused by exposure to sunlight can give rise to the appearance of skin cancer better known as carcinoma. Previously, a great variety of formulations and compositions for the treatment of this type of cancer have been commercialized, among which there are synthetic active ingredients commonly used for the treatment of this disease, but these mostly tend to cause injuries or side effects. Some of the most commonly used are known as pyridines, topical modulators and cyclo-oxygenase inhibitor compounds, among others. Therefore, the development and use of drugs composed of natural active agents, as well as in the U.S. Pat. No. 7,816,398 patent in which different compositions, pharmaceutical preparations and methods for the preparation of fatty alcohol conjugates and pharmaceutical agents useful in the treatment of cancer, viruses and psychiatric disorders. These natural compounds start from a fatty acid as an active agent obtained from the plants of the Annonaceae family better known as acetogenins, together with a mixture of surfactants that act as non-ionic agents and which are classified as those long chain fatty acids and their water insoluble derivatives. However, in this case, different salts, buffer solutions and preservatives such as chlorobutanol and parabens are used in the composition, which makes only a total amount of the formulation natural. Other drugs used for antitumor treatments such as the CN102552349 patent are obtained from the natural extracts of the Annona plant, such as annonacin. The formulation in this case is prepared to be injected or administered orally, which means that within the composition the addition of emollients, surfactants, vitamins, or preservatives is not taken into account, which if taken together with pH regulators in those formulas based on an anti-tumor cream for topical use. One of the main reasons for the use of 100% natural pharmacological formulations in the treatment of diseases in human skin, is to mitigate the occurrence of adverse effects. Occasionally, leaving aside the use of essential active agents to obtain a completely natural composition can be a risk as it is in the case of US20050079235A1 where a method is established to treat actinic keratosis by administering a topical pharmaceutical formulation of a polyphenol. The formulation is composed of preservatives such as benzoates, ascorbic acid, methylparabens, pH regulators such as citric acid, vitamin D and some natural oils or combinations of oils with water are added to form emulsions, but it is evident that no active agent is used to necessary obtain a natural cream, such as soursop extracts of the present invention that favor natural production. In this sense, WO03018036 describes the development of a new formulation based on natural and chemical extracts for the detection, treatment and cure of benign and malignant skin cancer and other skin lesions and tumors. This formulation is obtained from the mixture of various extracts such as Sanguinaria Canadensis Extract, Mexican Larrea Extract, Annona Muricata Extract (Soursop), Tabebuia Avellanedea Extract, among others. Similarly, the formulation has in its composition acids to maintain a certain pH and emulsifiers such as coconut oil and beeswax.

Other studies such as patent KR20100071476 describe a skin bleaching composition for the cosmetic industry where the active ingredient is Annona muricata or soursop extract, which is extracted with water, alcohol or a mixture thereof plus a solvent. Among the excipients are water, waxes, oils, surfactants, stabilizers, preservatives, vitamins and low alcohol content. Vegetable oils, beeswax, cetyl alcohols, fatty acids such as isopropyl palmitate and surfactants such as glycerin are added to the composition. However, the percentages in which all components are used differ entirely from the present invention.

Unlike what is reported in the state of the art, the composition of the present invention comprises a 100% natural formulation that specifically eliminates cancer cells and at the same time restores the properties of the skin without side effects, such as burning the skin, what happens with the commercial treatments that are currently used. On the other hand, the majority of the disclosed compositions do not correspond to a pharmaceutical set as defined in the composition of the present invention with a proven technical effect. The present invention achieves a stable and neutral composition that provides the specific technical elements that must be combined with the extract of Annona muricata, specifically of its leaves to achieve an anticancer effect.

### DESCRIPTION

The present invention relates to the development of a composition based on natural compounds that act as anticancer agents.

The main feature or technical element of this composition is a 100% natural compound that acts as an anti-cancer agent.

The composition of the present invention combines an active ingredient with a mixture of excipients specifically designed to form a stable formulation, with a neutral pH and with high effectiveness.

Particularly, the composition of the present invention comprises a natural active agent between 0.5% and 5%, from the natural extract of Annona muricata, up to 20% of a mixture of excipients and water.

The present invention provides a composition with a specific design that achieves a proven effect in the treatment of cancer lesions.

### BRIEF DESCRIPTION OF THE DRAWINGS

FIG. 1. Results of application of the composition of the invention with in vitro experiments using primary cancer cells.

FIG. 2. Results of application of the composition of the invention with in vitro experiments using primary cancer cells

### DETAILED DESCRIPTION OF THE INVENTION

The composition based on natural compounds of the present invention comprises between 0.5% and 5% of an extract of Annona muricata, up to 20% of a mixture of excipients composed of oils, emollients, emulsifiers, preservatives and vitamins and up to 80% of water.

The composition of the present invention comprises a natural active agent between 0.5% and 5%, from the natural extract of Annona muricata; preferably less than 1% of the natural extract of Annona muricata; more preferably between 0.5% and 0.8% of the natural extract Annona muricata.

For the purposes of the composition of the present invention, the term “extract” corresponds to a concentrate of plant components soluble in water or alcohol or in another suitable solvent, of one or more portions of a plant and may be in liquid or solid form.

For the same purposes, the Annona muricata extract of the present invention can be an organic extract or an aqueous extract.

In this same aspect of the invention, the natural extract comes from the leaves of Annona muricata. Particularly, the natural extract of Annona muricata leaves comprises acetogenins, alkaloids, phenols and others.

More particularly, the extract of the leaves of Annona muricata comprises compounds selected from the group of lactones, annohexocin, annomuricin A, B, C and E, annomutacin, annopentocins A, B and C, muricoreacin, gigantetronemine, murihexocin A and C, javoricin, isoquinolines, anonaine, anoniine, atherospermine, atheroreximine, lipids, gentisic acid, lignoceric acid, linoleic acid, stearic acid and/or combinations thereof and others.

According to this same aspect of the invention, the composition comprises up to 20% of a mixture of excipients composed of 0.8-8% of oils selected from the group of argan oil, almond oil, olive oil, wheat germ oil , rosehip oil, mineral oil, among others and/or combinations thereof and others; 8-25% of emollients selected from the group of allantoin, isopropyl palmitate, cetyl alcohol, among others, and/or combinations thereof and others; 0.2-7% of emulsifiers selected from the group of arlacel 165, tween 20, beeswax, aloe vera, among others, and/or combinations thereof; 0-5% of selected preservatives of the group of emulgin B2, emulgin VL 75, eutanol G, euxyl K300, sodium benzoate, methyl methyl paraben, sodium silicate, butyl paraben, 1,3 propanediol, ammonium propionate, among others, and combinations of them and others; and 0.5-2% of vitamins selected from the group of vitamin A, C, E, B12, B5, D3 TYPE 100, K1, among others and/or combinations thereof and others.

In this same aspect, the composition of the present invention comprises water up to 80%.

In this same aspect, the pH of the composition of the present invention is kept neutral.

Optionally, the composition of the present invention comprises an additional natural component selected from the group of seaweed extract, arnica, marigold extract, horse chestnut extract, Asiatic scintilla extract, horsetail extract, Ginkgo biloba extract, Hammamelis spp, among others, and combinations thereof and others.

Optionally, the composition of the present invention comprises 0-2% of pH regulator selected from the group of as citric acid, ascorbic acid, nitric acid or triethanol amine to preserve the pH between 5-7.

### Preferred Embodiments

In the preferred aspect of the invention, the composition comprises a natural active agent between 0.5% and 1%, from the natural extract of Annona muricata; preferably less than 1% of the natural extract of Annona muricata; more preferably between 0.5 and 0.8% of the natural extract Annona muricata.

In this same preferred aspect, the composition of the invention comprises between 0.5 and 0.8 of natural extract of Annona muricata leaves. Even in this preferred aspect of the invention, the composition comprises between 0.5 and 0.8 of a mixture of specific chemical compounds from Annona muricata leaves.

In this same aspect of the invention, the composition comprises up to 20% of a mixture of excipients composed of 0.8-8% of oils selected from the wheat germ oil group; 8-25% isopropyl palmitate; 0.2-7% of emulsifiers selected from the group of arlacel 165, tween 20, beeswax, aloe vera, among others, and/or combinations thereof; 0-5% of selected preservatives of the group of emulgin B2, emulgin VL 75, ethanol G, euxyl K300, sodium benzoate, methyl methyl paraben, sodium silicate, butyl paraben, 1,3 propanediol, ammonium propionate, among others, and combinations of them and others; and about 2% of vitamin E. In this same aspect, the composition of the present invention comprises water up to 80%. In this same aspect, the pH of the composition of the present invention is maintained 5 and 7.

### EXAMPLES

### 1.7.1 Selection of the Active Agent of the Composition of the Invention Using In Vitro Experiments

We assessed the in vitro impact of three natural compounds (A, B and C) on the growth/proliferation and apoptosis (cell death) of stem and progenitor primary cells isolated from cancer patient cells.

First, extracts of natural compounds A, B, and C were obtained by steam distillation. Compound A is from a European native plant, compound B is from a Colombian native plant, which corresponds to the natural active agent of the invention and compound C from a plant native to southeastern India. Extracts at an adequate concentration of the molecules of the main assets contained in plants, in order to make their action more effective.

Subsequently, the extracts were diluted in different concentrations to study the effect of the 3 compounds A, B and C with respect to the growth and clonogenic properties of cancer cells (n=6). For this purpose, an assay was applied that consists in measuring the ability of stem/progenitor cells to grow, divide and give rise to carcinogenic colonies in a semi-solid medium. Short-term exposure of primary cancer cells (n=6) in a liquid culture for 48 hours without or with increasing doses of compounds A and B completely abolished the growth of cancer stem/progenitor cells in vitro, even at lowest concentration (FIG. 1). The effect of progressive doses of compounds A, B and C on the apoptosis (cell death) of cancer cells (n=4) was continued by monitoring the percentage of cells stained with Annexin V positive. It was found that very low doses of compounds A, B and C are sufficient to significantly induce a massive apoptosis or cell death of cancer stem cells (n=4). These pre-clinical results in primary cells strongly suggest that the three natural compounds A, B and C have a very potent bioactivity and strong anti-cancer activity in vitro.

In addition, similar results were observed using several other cancer cells using the same experiments to measure the ability of stem/progenitor cells to grow, divide and grow cancer colonies.

Based on all these results, out of the three natural compounds, compound B was selected since it showed or displayed the highest potential for anti-cancer activity.

### 1.7.2 Design of the Composition of the Invention With Different Concentrations of the Natural Active Agent

The next step was the elaboration of a neutral base cream. To this cream, different concentrations of the extract were added to assess its stability and its potential to decrease the presence of actinic keratosis. Samples with different concentrations are evaluated (which we will call samples 1, 2, 3, 4, 5 and 6).

The samples have a light beige color and are stable in a centrifugal test at 3200 rpm, for 20 minutes. Later they are introduced in an oven at 35° C. to see how susceptible they are to be degraded. Sample 5 shows an increase in color intensity, showing its long-term instability and a shorter lifespan than the other samples. Next, the pH of the remaining samples is evaluated, obtaining a range between 5.1 and 6.1.

Different samples of each concentration are stored under different conditions in order to evaluate their stability against different stimuli, the conditions established with:

1) Oven at 35° C.,

2) On shelves (cool and dry place) and

3) Refrigeration (4° C.).

After 3 and 5 months the same characteristics are evaluated: coloration, separation, pH obtaining the same results as in the first analysis.

These results provide that the shelf life of the samples is at least 9 months on the shelf.

### 1.7.3 Test With Patients With the Composition of the Present Invention

The next step was to do a short 6 week test of the different samples with several patients (n=4) that had cancerous skin lesions.

The evolution of the patients shows that creams 3 and 4 seem to have the same efficacy in the treatment, so it is decided to study cream 3 in depth in a larger number of patients.

For this last part of the study, 12 patients with clinical diagnosis of cancerous lesions on their face and scalp were selected. These patients, 10 male and 2 female; whose ages fluctuated between 65 and 83 years of age, without taking into consideration any of their previous treatments, but without dermatological management for 2 months prior to the start of this test.

Each patient was given cream 3, which corresponds to the composition of the present invention, based on the compound of a Colombian native plant, with a standard dosage of topical application 2 times a day (morning and night) in an amount of 2mg per square centimeter approximately and the use of sunscreen. The clinical condition was registered, and the respective photographic records were taken at 2, 4, 8 and 12 weeks. The results of these 12 patients evaluated by the patient and dermatologist in all patients have been excellent. The response to therapy was assessed as satisfactory, since patients responded positively to treatment. The texture of their skin improved, the scales and the roughness of the lesions decreased considerably and even many disappeared despite the short observation period, and in general the patients expressed feeling well with an improvement of the treated lesions and the texture in general of their skin. There were no adverse effects in any of the patients. Based on all the above, it is concluded that the composition of the invention limits and prevents the development of cancer lesions, but controlled studies have yet to be done to corroborate the results obtained in this preliminary study.

1. A composition based on natural compounds comprising between 0.5% and 5% of an extract of Annona muricata, up to 20% of a mixture of excipients composed of oils, emollients, emulsifiers, preservatives and vitamins and up to 80% of water.

2. The composition based on natural compounds according to claim 1, comprising less than 1% of the natural extract of Annona muricata.

3. The composition based on natural compounds according to claims 1 to 2, comprising between 0.5 5 and 0.8% of the natural extract Annona muricata.

4. The composition based on natural compounds according to claims 1 to 3, comprising between 0.5 5 and 0.8% of the natural extract of Annona muricata leaves.

5. The composition based on natural compounds according to claim 1, comprising up to 20% of a mixture of excipients composed of 0.8-8% of oils selected from the group of argan oil, almond oil, oil olive, wheat germ oil, rosehip oil, mineral oil, among others and/or combinations thereof and others; 8-25% of emollients selected from the group of allantoin, isopropyl palmitate, cetyl alcohol, among others, and/or combinations thereof and others; 0.2-7% of emulsifiers selected from the group of arlacel 165, tween 20, beeswax, aloe vera, among others, and/or combinations thereof; 0-5% of selected preservatives of the group of emulgin B2, emulgin VL 75, eutanol G, euxyl K300, sodium benzoate, methyl methyl paraben, sodium silicate, butyl paraben, 1,3 propanediol, ammonium propionate, among others, and combinations of them and others; and 0.5-2% of vitamins selected from the group of vitamin A, C, E, B12, B5, D3 TYPE 100, K1, among others and/or combinations thereof and others.

6. The composition based on natural compounds according to claim 1, wherein the pH is kept neutral.
